(19) United States
(12) Patent Application Publication
(10) Pub. No.: US 2015/0305795 A1
(43) Pub. Date: Oct. 29, 2015

(54) ELECTROSURGICAL PLASMA APPARATUS AND SYSTEM

(71) Applicant: GYRUS MEDICAL LIMITED, Cardiff (GB)

(72) Inventors: Kelvin John VARNEY, Abergavenny (GB); Marno NAGTEGAAL, Cardiff (GB); Teo Heng Jimmy YANG, Cardiff (GB)

(21) Appl. No.: 14/431,672

(22) PCT Filed: Oct. 3, 2013
(86) PCT No.: PCT/GB2013/052582

§ 371 Date: Mar. 26, 2015

(30) Foreign Application Priority Data
Oct. 4, 2012 (GB) 1217781.2

(51) Int. Cl. A61B 18/04

(52) U.S. Cl. CPC A61B 18/042; A61B 2018/00583

(57) ABSTRACT

An electrosurgical apparatus for coagulating tissue includes an elongated tube having a tubular wall and a proximal end and a distal end, and constituting a conduit though which ionisable gas can be supplied to the distal end of the tube. The tube includes one or more apertures in the tube such that the ionisable gas is capable of exiting the tube in the region of the distal end of the tube. A braided tubular component is associated with the wall of the tube, and is connected to a source of electrosurgical energy, such that the braided tubular component can form part of an electrode assembly for ionising the ionisable gas exiting the one or more apertures.

### TECHNICAL FIELD

This invention relates to an electrosurgical apparatus and system and in particular to the non-contact coagulation of tissue using an ionisable gas such as argon.

### BACKGROUND TO THE INVENTION AND PRIOR ART

Argon beam coagulators have been known for many years, and examples are given in U.S. Pat. Nos. 4,040,426, 5,720,745, 6,039,736 and 6,197,026. The first example is an end-effect instrument, in which the ionised gas exits through the end of the instrument, while the latter two examples are directed at side-effect instruments, in which the ionised gas exits the instrument though an aperture in the side of the instrument. Such instruments are often referred to as APC instruments (Argon Plasma Coagulation).

### SUMMARY OF THE INVENTION

Embodiments of the invention attempt to provide an instrument which is more versatile than any of the instruments in the prior art, and accordingly one aspect of the invention resides in an electrosurgical apparatus for coagulating tissue, comprising an elongated tube having a tubular wall and a proximal end and a distal end,

a conduit though which ionisable gas can be supplied to the distal end of the tube, the tube including one or more apertures in the tube such that the ionisable gas is capable of exiting the tube in the region of the distal end of the tube,

a braided tubular component associated with the wall of the tube, and

a connector for connecting the braided tubular component to a source of electrical energy, such that the braided tubular component can form part of an electrode assembly for ionising the ionisable gas exiting the one or more apertures.

The braided tubular component forms part of an electrode assembly in that it can either constitute a lead forming an electrical path between the connector and an electrode element, or may alternatively itself constitute an electrode element. In the first alternative, the electrode assembly constitutes the braided tubular component plus a separate electrode element, whereas in the second alternative the electrode assembly merely constitutes the braided tubular component.

The braided tubular component may be “associated” with the wall of the tube in that the tubular wall comprises an inner surface and an outer surface, and the braided tubular component is located adjacent the inner surface of the tubular wall. Alternatively, the braided tubular component may be “associated” with the wall of the tube in that the braided tubular component is embedded in the tubular wall. In one convenient construction, the braided tubular component is an inner layer in a laminate structure comprising the braided tubular component and a plurality of layers of electrically insulating material. Whichever arrangement is employed, the braided component is a tubular component extending around the circumference of the elongated tube, as oppose to a braided wire which runs along a single path in or adjacent the elongated tube. In this way, the braided tubular component is present around 360° with respect to the elongated tube, so as to be able to provide an electrical presence in whichever radial direction is required.

An insulative sleeve may be provided arranged to insulate the braided tubular component from the conduit other than in regions where the braided tubular component is to ionise the ionisable gas. In this respect, in some embodiments the insulative sleeve does not extend about or around the apertures to allow the braided tubular component to form part of the electrode assembly for ionising the ionisable gas exiting the one or more apertures.

According to one convenient arrangement, the one or more apertures includes an aperture at the distal end of the tube. This may be provided by the tube having an open end face constituting the aperture at the distal end of the tube, or alternatively by the tube having a distal end face, the aperture at the distal end of the tube being formed in the distal end face. With either construction, the braided tubular component is preferably exposed at the distal end of the tube so as to form the electrode element for ionising the ionisable gas exiting the aperture.

Where the braided tubular component does not constitute the electrode itself, but merely a lead for a separate electrode element, the electrode element is conveniently a separate annular ring positioned at the distal end of the tube and electrically connected to the braided tubular component. This allows the provision of a solid annular ring as an electrode, which may provide more resistance to wear and erosion from the ionisation of the gas, as compared to the braided component. Other electrode elements of different shapes can be envisaged as alternatives to an annular ring.

According to an alternative arrangement, the one or more apertures conveniently includes one or more side apertures in the wall of the tube. In this arrangement, the braided tubular component is conveniently exposed in the region of the one or more side apertures so as to form the electrode element for ionising the ionisable gas exiting the aperture. Due to the tubular nature of the braided component, it is ensured that a portion of the braided component will always be available to provide the electrode element regardless of the number of apertures or their radial positioning.

In another aspect the invention further resides in an electrosurgical system including an electrosurgical generator, a source of ionisable gas, and an electrosurgical apparatus as described above. The electrosurgical generator provides an electrical RF signal, as is known in the art. In one arrangement, the system also includes a patient return electrode connected to the electrosurgical generator, such that the electrosurgical apparatus is effectively a monopolar apparatus. Alternatively, the electrosurgical apparatus also includes a return electrode connected to the electrosurgical generator, such that the electrosurgical apparatus is effectively a bipolar apparatus. In this bipolar arrangement, the return electrode conveniently also comprises a braided tubular component. In such a system, the return electrode is preferably a layer in a laminate structure comprising the braided tubular component and a plurality of layers of electrically insulating material.

In another aspect the invention further resides in an electrosurgical apparatus for coagulating tissue, comprising:

an elongated tube having a tubular wall and a proximal end and a distal end,

a conduit though which ionisable gas can be supplied to the distal end of the tube, the tube including one or more apertures in the tube such that the ionisable gas is capable of exiting the tube in the region of the distal end of the tube,

a first braided tubular component associated with the wall of the tube,

a second braided tubular component associated with the wall of the tube, and

a connector for connecting the first and second braided tubular components to a source of electrical energy, such that the first and second braided tubular components can form part of a bipolar electrode assembly for ionising the ionisable gas exiting the one or more apertures.

### BRIEF DESCRIPTION OF THE DRAWINGS

Embodiments of the invention will now be further described, by way of example only, with reference to the accompanying drawings, in which:

FIG. 1 is a schematic view of an electrosurgical system in accordance with the present invention,

FIG. 2 is a schematic part-sectional view of the tip of an electrosurgical instrument used as part of the electrosurgical system of FIG. 1,

FIG. 3 is a schematic part-sectional view of the tip of an alternative embodiment of electrosurgical instrument according to the invention,

FIG. 4 is a schematic part-sectional view of the tip of another alternative embodiment of electrosurgical instrument according to the invention,

FIG. 5 is a side view of the tip of another alternative embodiment of electrosurgical instrument according to the invention,

FIG. 6A is a schematic part-sectional view of the tip of a further alternative embodiment of electrosurgical instrument according to the invention,

FIG. 6B is an end view of the electrosurgical instrument of FIG. 6A,

FIG. 7A is a schematic part-sectional view of the tip of a further alternative embodiment of electrosurgical instrument according to the invention,

FIG. 7B is an end view of the electrosurgical instrument of FIG. 7A,

FIG. 8A is a schematic part-sectional view of the tip of a further alternative embodiment of electrosurgical instrument according to the invention,

FIG. 8B is an end view of the electrosurgical instrument of FIG. 8A,

FIG. 9 is a schematic part-sectional view of the tip of a further alternative embodiment of electrosurgical instrument according to the invention, and

FIG. 10 is a schematic sectional view of the tip of a further alternative embodiment of electrosurgical instrument according to the invention.

### DESCRIPTION OF THE EMBODIMENTS

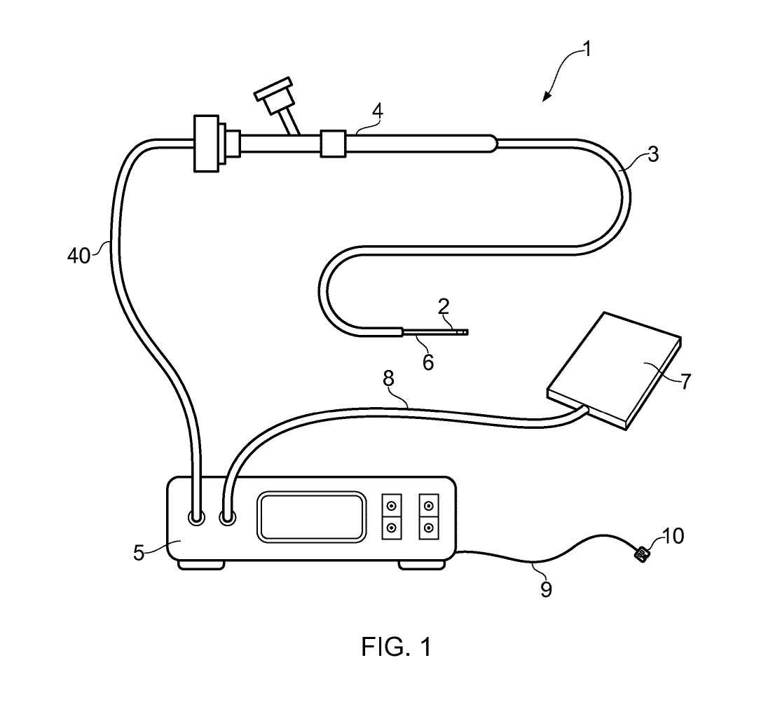

Referring to FIG. 1, an APC system comprises an instrument shown generally at 1, and comprising a working instrument 2 disposed through an elongate flexible sheath 3 extending from an endoscope 4. The working instrument 2 is connected to a radio frequency signal generator 5 via flexible cable 40, the generator 5 also including a source of argon gas (not shown) which is also supplied through the cable 40. The working instrument 2 comprises an elongate tube 6 to be described in more detail subsequently. A patient return plate 7 is also connected to the generator 5 by means of cable 8. The generator 5 is connected to a source of power by lead 9 and plug 10.

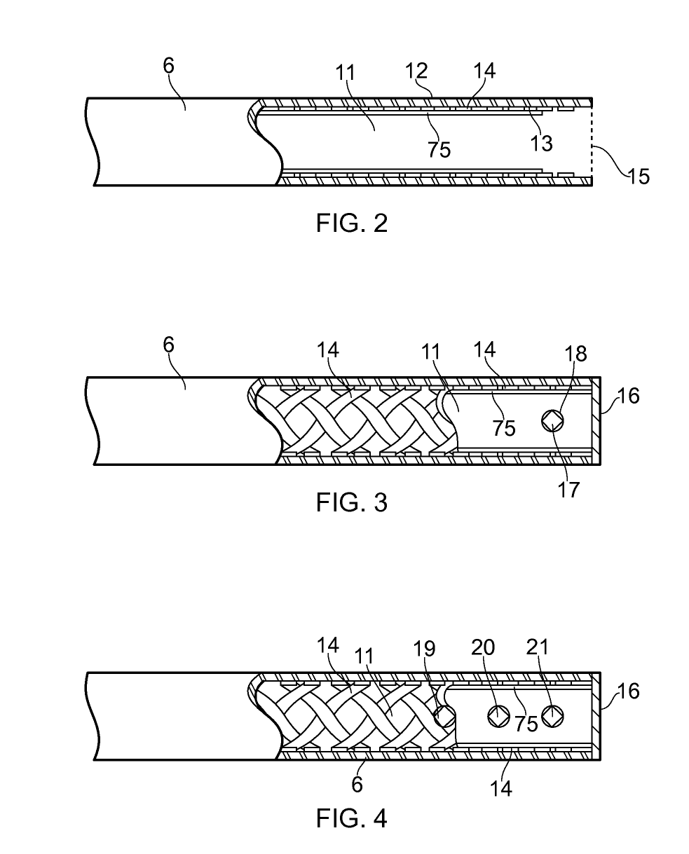

FIG. 2 shows the distal end of the working instrument 2. The elongate tube 6 is hollow so as to form a gas conduit 11 therein, and includes an outer wall 12 and an inner wall 13. The tube 6 is formed of an electrically insulating material such as a durable plastics material. A tubular braid 14 of electrically conductive material is located in the gas conduit 11 adjacent the inner wall 13 of the tube 6. An insulative sleeve 75 is provided within the tubular braid, which in this embodiment extends along the majority of the length of the braid, but stops short of the distal end of the tube 6, such that the tubular braid is exposed adjacent an aperture 15. The tubular braid 14 may be connected to the radio frequency generator by means of a lead (not shown) running the length of the flexible sheath 3, or alternatively the tubular braid may itself extend along the sheath forming an electrical connection to the generator 5.

The tube 6 has an open distal end forming the aperture 15 for the argon gas to exit the tube 6. In use, the gas is supplied along the conduit 11, and a high voltage radio frequency waveform is supplied to the tubular braid 14. The braid 14 acts as an electrode to ionise the argon gas as it exits the aperture 15. Due to the insulative sleeve 75 stopping short of the distal end of the tube 6, ionisation occurs in the region of the aperture, where the tubular braid is exposed to the gas conduit 11 in the region where the insulative sleeve 75 does not extend.

FIG. 3 shows an alternative instrument in which the tube 6 has a closed end face 16. However, an aperture 17 is provided in the side of the tube 6 such that the gas can exit the tube orthogonally to the longitudinal axis of the tube. The tubular braid 14 and insulative sleeve 75 are such that a portion 18 of the tubular braid is exposed adjacent the aperture 17, such that the braid can act as an electrode to ionise the gas exiting the aperture 17.

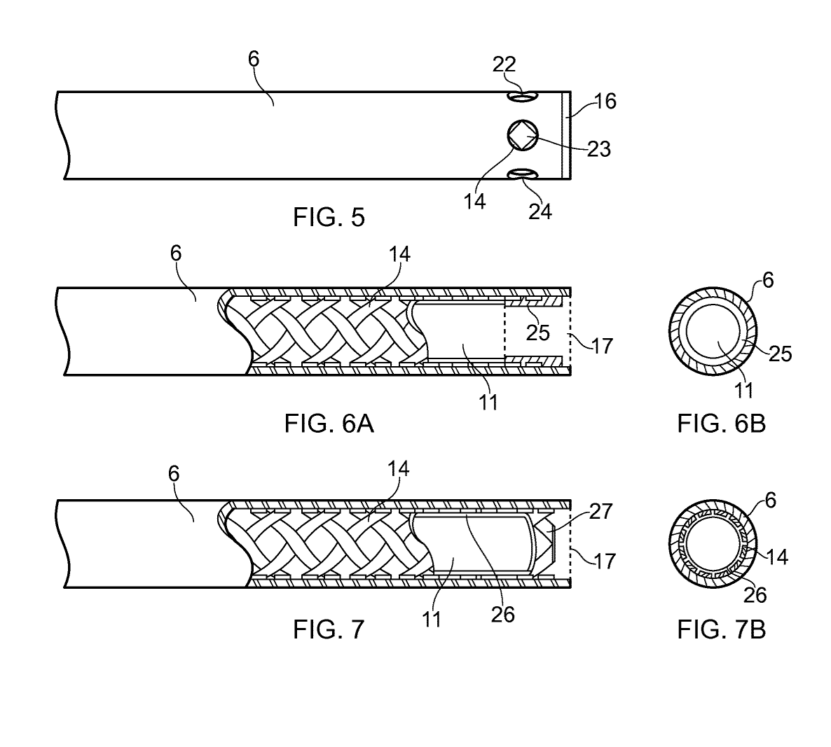

FIG. 4 is similar to FIG. 3, except that a plurality of side apertures 19, 20 & 21 are provided along the longitudinal axis of the tube 6. The construction of the braid 14 and the insulative sleeve 75 is such that a portion of the braid is exposed adjacent each aperture, whatever its longitudinal position. FIG. 5 shows an alternative instrument, in which a plurality of side apertures 22, 23, 24 etc. are provided at the same longitudinal position along the tube 6 but spaced around the circumference thereof so as to allow the gas to exit at different radial positions around the tube. Once again, whatever the radial position of the apertures 22, 23, 24, the construction of the braid 14 and insulative sleeve is such that a portion of the braid is exposed adjacent each aperture to allow for gas ionisation.

FIGS. 6A & 6B show an alternative instrument, in which the tubular braid 14 acts not as an electrode but as a lead to a separate electrode element in the form of an annular ring 25. The insulative sleeve 75 extends over the tubular braid, but not the annular ring. The ring 25 is connected to the braid 14 at the distal end of the tube 6, and acts as the electrode to ionise the argon gas travelling along the conduit 11. The tube 6 has an open distal end providing an aperture 17, as in the instrument of FIG. 3.

FIGS. 7A & 7B show an instrument which is provided with an additional inner layer 26 of electrically insulating material, such that the tubular braid 14 is the middle layer in a laminate structure comprising the tube 6, the braid 14 and the inner layer 26. The inner layer 26 stops just short of the distal end of the tube 6, such that the braid 14 is exposed at its distal end. In this way, the exposed portion 27 of the braid 14 acts as an electrode to ionise the argon gas exiting the end of the tube through the aperture 17.

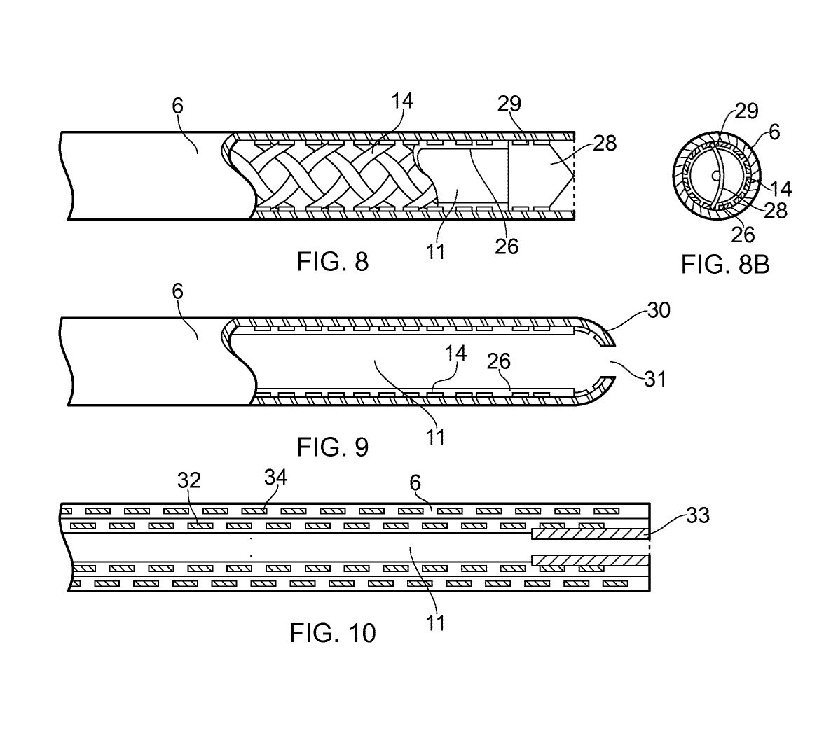

FIGS. 8A & 8B show a similar arrangement in which an inner layer 26 of insulation is provided over the braid 14, but in which a separate electrode element 28 acts as the electrode rather than the braid itself. The electrode element 28 is in the form of a shaped plate, and is electrically connected to the braid 14 by means of the sharp edges 29 of the electrode element 28 cutting through the inner layer 26 in order to make contact with the braid 14. The shaped plate provides a durable electrode with a controlled ignition point for the ionised gas flowing along the conduit 11.

FIG. 9 shows a further variation, in which an inner layer 26 of insulation is once again provided over the braid 14. As in FIG. 7, the inner layer 26 stops just short of the distal end of the tube 6, such that the braid 14 is exposed at its distal end to act as an electrode. However, rather than a fully open end face, the tube has a shaped distal end 30 with an aperture 31 formed therein. In this way, ionised argon gas is constrained to flow through a relatively small diameter orifice when it exits the tube 6, so as to form a fine and focussed beam of ionised gas.

Finally, FIG. 10 shows a bipolar version of the instrument 1, in which the patient return plate 7 is replaced with an electrode carried within the tube 6. In FIG. 10, the tube 6 comprises an inner tubular braid 32 forming the electrical connection to an annular electrode 33 located at the distal end of the tube. An outer tubular braid 34 is also embedded within the tube 6, coaxially located and spaced from the inner braid such that the insulating material of the tube 6 isolates one braid from the other. The outer braid 34 is also connected to the electrosurgical generator 5, such that it can act as a return electrode for the annular electrode 33. As argon gas flows along the conduit 11, it is ionised by the annular electrode 33, the electric circuit being completed by capacitive coupling to the outer braid 34 present within the tube 6. Alternatively, a portion of the outer braid can be exposed (not shown) to provide a direct connection for the completion of the circuit. Whichever method is used, the instrument 1 acts as a bipolar instrument, with the outer braid 34 acting as a return electrode for the annular electrode 33.

Those skilled in the art will appreciate that other constructions can be envisaged without departing from the scope of the present invention. For example, the number, location and shape of the apertures can be varied, as can the shape of the electrode element, if one is used in addition to the tubular braid. The instrument can be made rigid or flexible, depending on the intended use, and different versions of the system can be envisaged for endoscopic, laparoscopic or open surgical use.

1. An electrosurgical system for coagulating tissue, the system including:
a) an electrosurgical generator;
b) a source of ionisable gas, and
c) an instrument for coagulating tissue, the instrument comprising:
an elongated tube having a tubular wall and a proximal end and a distal end,
a conduit though which ionisable gas can be supplied in use to the distal end of the tube, the tube including one or more apertures in the tube such that in use the ionisable gas exits the tube in the region of the distal end of the tube,
a braided tubular component associated with the wall of the tube, and
a connector for connecting the braided tubular component to the electrosurgical generator, such that the braided tubular component forms part of an electrode assembly for ionising in use the ionisable gas exiting the one or more apertures.

2. A system according to claim 1, wherein the braided tubular component constitutes a lead forming an electrical path between the connector and an electrode element.

3. A system according to claim 1, wherein the braided tubular component constitutes an electrode element.

4. A system according to claim 2, wherein the tubular wall comprises an inner surface and an outer surface, and the braided tubular component is located adjacent the inner surface of the tubular wall.

5. A system according to claim 1, and further comprising an insulative sleeve arranged to insulate the braided tubular component from the conduit other than in regions where the braided tubular component is to ionise the ionisable gas.

6. A system according to claim 5, wherein the insulative sleeve does not extend about or around the apertures to allow the braided tubular component to form part of the electrode assembly for ionising the ionisable gas exiting the one or more apertures.

7. A system according to claim 2, wherein the braided tubular component is embedded in the tubular wall.

8. A system according to claim 7, wherein the braided tubular component is an inner layer in a laminate structure comprising the braided tubular component and a plurality of layers of electrically insulating material.

9. A system according to claim 1, wherein the one or more apertures includes an aperture at the distal end of the tube.

10. A system according to claim 9, wherein the tube has an open end face constituting the aperture at the distal end of the tube.

11. A system according to claim 9, wherein the tube has a distal end face, the aperture at the distal end of the tube being formed in the distal end face.

12. A system according to claim 9, when dependent on claim 3, wherein the braided tubular component is exposed at the distal end of the tube so as to form the electrode element for ionising the ionisable gas exiting the aperture.

13. A system according to claim 1, wherein the one or more apertures includes one or more side apertures in the wall of the tube.

14. A according to claim 13, wherein the braided tubular component constitutes an electrode element and is exposed in the region of the one or more side apertures so as to form the electrode element for ionising the ionisable gas exiting the aperture.

15. (canceled)

16. A system according to claim 1, wherein the system also includes a patient return electrode connected to the electrosurgical generator.

17. A system according to claim 1, wherein the electrosurgical apparatus also includes a return electrode connected to the electrosurgical generator.

18. A system according to claim 17, wherein the return electrode comprises a braided tubular component.

19. A system according to claim 18, wherein the return electrode is a layer in a laminate structure comprising the braided tubular component and a plurality of layers of electrically insulating material.

20. A system according to claim 1, wherein the instrument includes:
a second braided tubular component associated with the wall of the tube, and
a connector for connecting the first and second braided tubular components to a source of electrical energy, such that the first and second braided tubular components can form part of a bipolar electrode assembly for ionising the ionisable gas exiting the one or more apertures.
